(19) United States
(12) Patent Application Publication
(10) Pub. No.: US 2004/0043059 A1
(43) Pub. Date: Mar. 4, 2004

(54) Anti-wrinkle patch

(76) Inventor: David Canny, Santa Clara, CA (US)

Correspondence Address: DAVID CANNY, 2275 S Bascom Ave #802, Santa Clara, CA, 95051, US

(21) Appl. No.: 10/232,751

(22) Filed: Aug. 31, 2002

(51) Int. Cl. A61K 9/70

(52) U.S. Cl. 424/443

(57) ABSTRACT

Anti-Wrinkle Patch with a thin flat flexible sheet die cut to a shape that fits generally in the area directly below the user's eye. The flat sheet has a non-allergic adhesive substrate on one side. The flat sheet perimeter or portion of thereof has a non-adhesive convex shaped frame. A preferred embodiment includes the adhesive substrate containing additional anti-wrinkle ingredients such as anti-oxidants or other agents that claim to reduce wrinkles.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of cosmetic aids and more specifically to an anti-wrinkle patch.

Aging and aging of the face result in reduced elasticity of the skin and wrinkles in the skin. A person can have some control over the acceleration of the wrinkle effect by maintaining good habits of nutrition and exercise, and by reducing exposure to the sun. Improvements can also be gained by application of various cosmetic skin care products and even cosmetic surgery. One area of the face most effected by wrinkles is under the eyes. When one lies down and\or sleeps through the night the cheeks on one's face fall back around the area of the eyes. This results in wrinkles under the eyes that are particularly noticeable in the morning after waking up from sleep. The depth of wrinkles usually reduce during the day because in the upright position gravity causes the cheeks to pull down on the skin around the eyes thereby reducing the wrinkles. Morning eye wrinkles also result from one's face being squeezed against the pillow when one sleeps on their side.

One method of reducing the effect of wrinkles around the eyes has been to employ a flat patch such as the Face Lift patches manufactured by University Medical Products Inc. of Irvine Calif. These patches are flat and flexible and contain anti-wrinkle collagen along with the adhesive material needed to adhere the patch to the user's face. Some patents such as U.S. Pat. Nos. 6,296,869, 6,280,765, 6,180,133, and 6,191,339 provide compositions for improving and reducing wrinkles and use a patch as a method of application of these compounds. Patent Des. 355485 by Almond and D440315 by Hassenbein show shapes of patches and pads respectively that are thought to best fit on various facial contours. D440315 by Hassenbein as stated in its patent title is more of a pad for applying anti-wrinkle compounds than a patch that fixes the skin in place.

There is a deficiency in the prior technology however, in that the relatively sharp perimeter edges of the existing patches can produce new wrinkles around it's edge or deepen an existing wrinkle due to the edge digging in to the skin. My experiments show that even without the use of anti-wrinkle agents such as anti-oxidants and the like, my patch design is effective because it keeps the skin underneath the adhesive section fixed in place when one lies down and\or sleeps, while unlike the prior art, it does not create additional wrinkles or deepen existing wrinkles due to the edge of the patch.

### BRIEF SUMMARY OF THE INVENTION

The primary object of the invention is to provide an anti-wrinkle patch that significantly reduces wrinkles and is particularly suitable for treating wrinkles under the eyes.

Another object of the invention is to provide a flexible anti-wrinkle patch with a convex shaped frame that can be applied by the user and is designed in a way that does not create additional wrinkles or deepen existing wrinkles along the edges at the perimeter of the device.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed an Anti-Wrinkle Patch comprising: a thin flat flexible sheet die cut to a shape that fits generally in the area directly below the user's eye, said flat sheet having a non-allergic adhesive substrate on one side, and said flat sheet perimeter or portion of thereof having a non-adhesive convex shaped frame.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a front view of the anti-wrinkle patch of the present invention as worn on a person.

FIG. 2 is a side view of the anti-wrinkle patch of the present invention as worn on a person.

FIG. 3 is a side section view of the present invention.

FIG. 4 is an exploded view of an alternate embodiment of the present invention.

FIG. 5 is a side section view of the alternate embodiment of the present invention.

FIG. 6 is a side view of a person using a patch product that is currently for sale in the marketplace.

FIG. 7 is a front view of an alternate embodiment of the present invention.

### DETAILED DESCRIPTION OF THE PREFERRED EMBODIMENTS

Detailed Description of the Preferred Embodiments are provided herein. It is to be understood however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

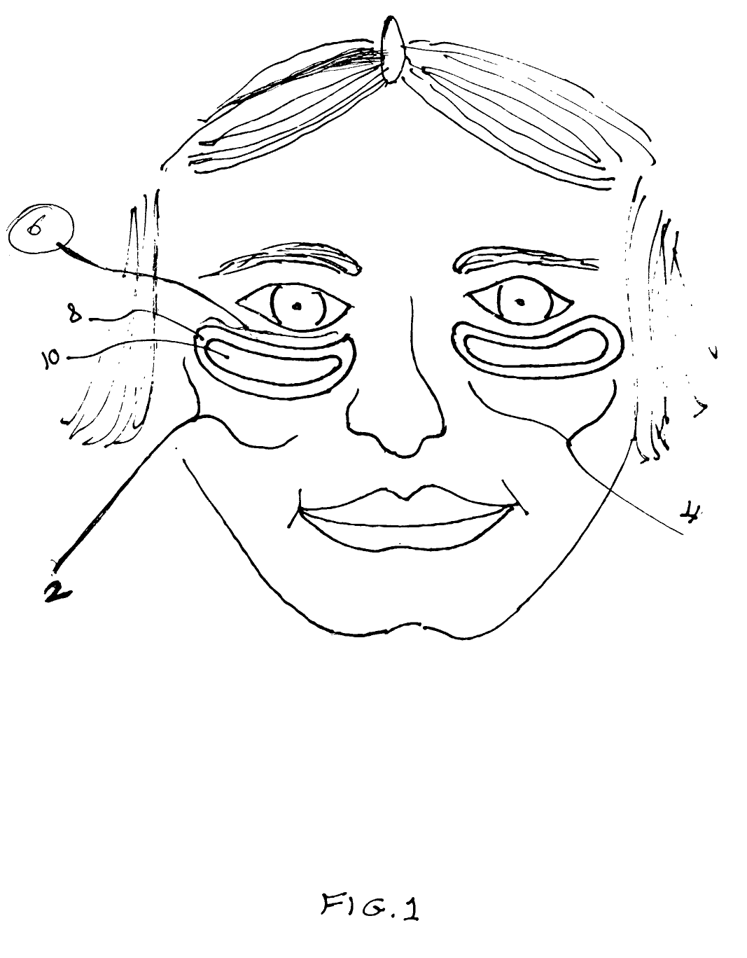

Referring now to FIG. 1 we see a front view of a person wearing the anti-wrinkle patch 2, 4, of the present invention.

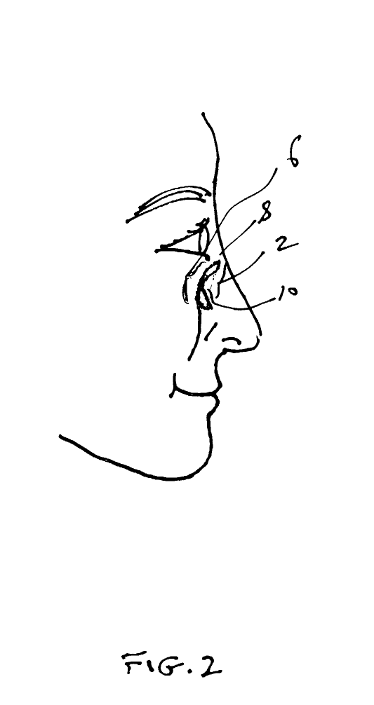

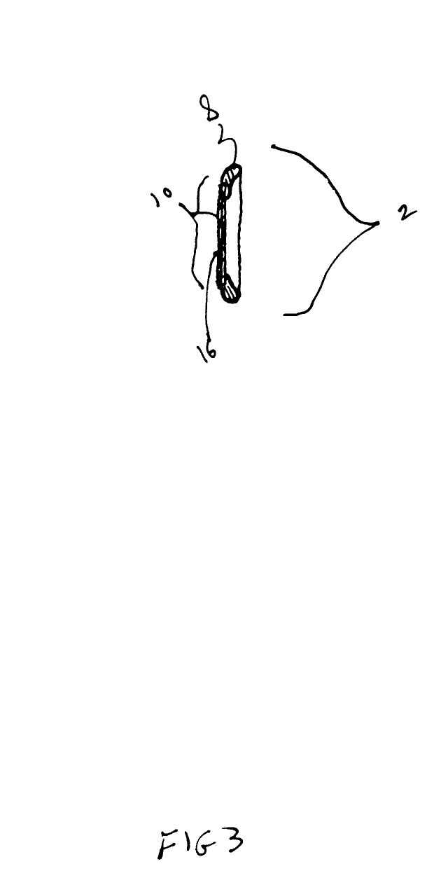

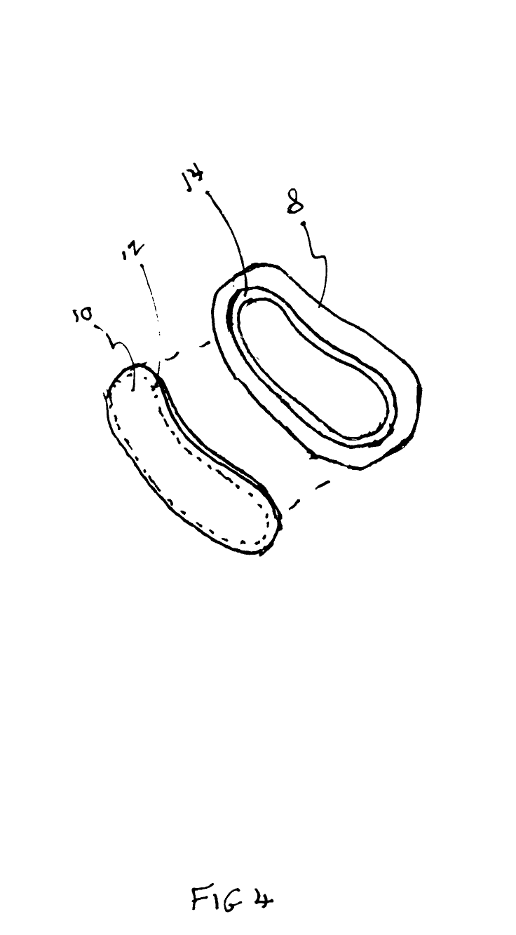

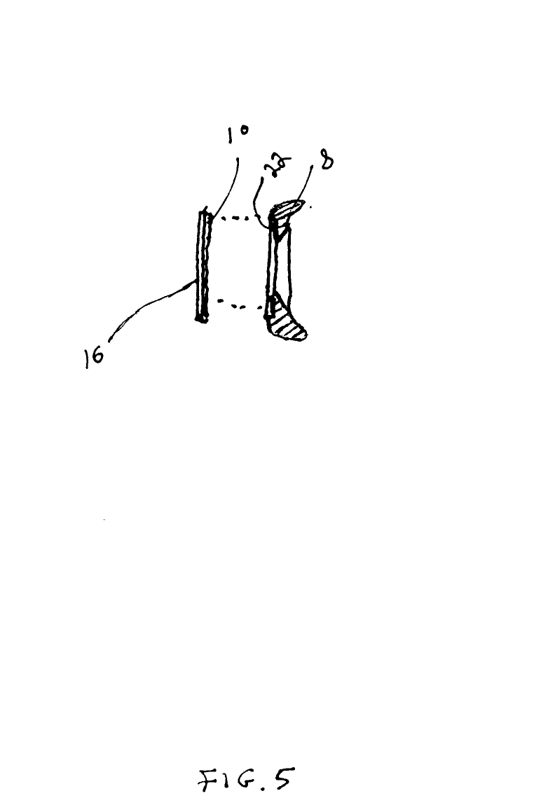

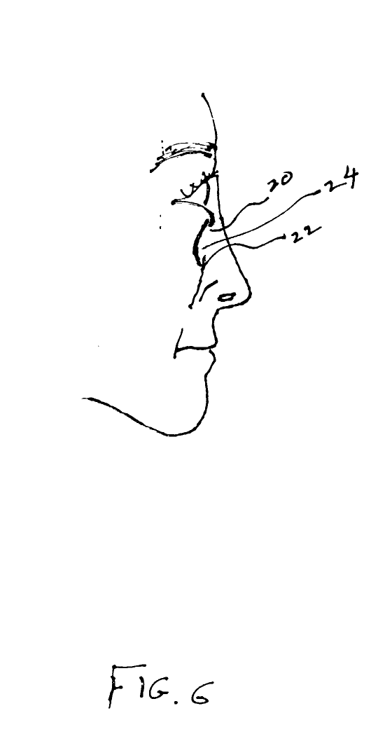

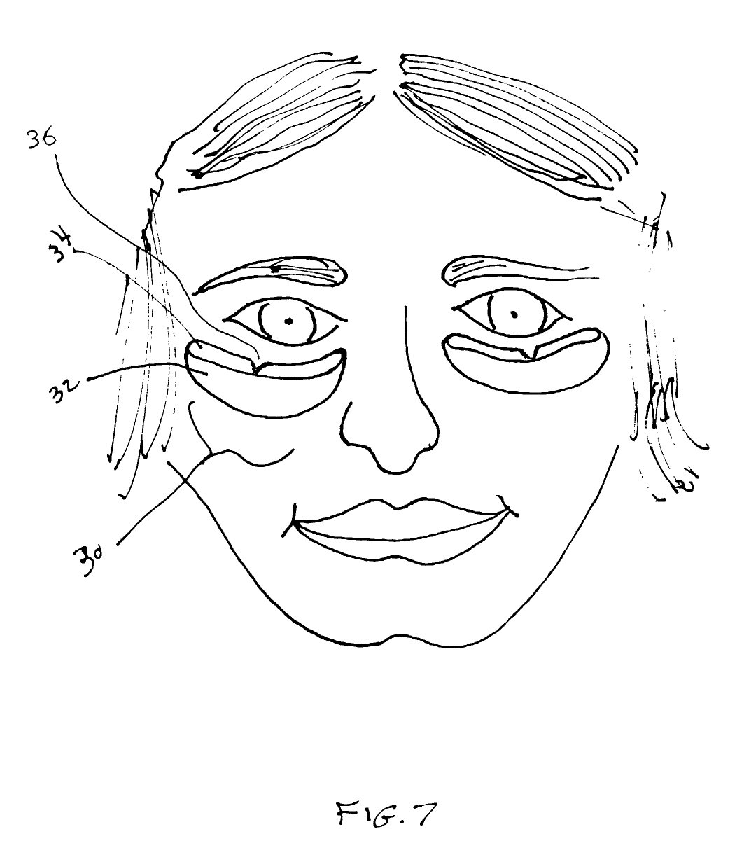

The patches are placed below the person's eyes and act to hold the skin in position so that when a person when a person lies down and\or sleeps, the area under the eyes 6 does not become compressed and wrinkled. The patches are comprised of a convex shaped non-adhesive frame 8 adhered to a thin film sheet 10. The sheet 10 has an adhesive material bonded to its underside so that it can irremovably adhere to the user's skin. The adhesive 16 as shown in FIG. 5 can also include skin care products such as collagen or anti-oxidants for additional wrinkle reduction. There is a smooth transition from the non-adhesive frame 8 to the adhesive 16 so as not to distort the skin under this joint. Frame 8 can be made of non-allergic flexible or non-flexible plastic or other known resilient, skin friendly material. The rounded edges of frame 8 do not cause distortion of the skin immediately below the eye as shown in FIG. 2. Other patches 24 available on the market today, as shown in FIG. 6, do not include the frame 8 and can cause distortion of the skin at areas 20, 22. FIG. 3 shows a side section view of the present invention 2. The frame member 8 of the preferred embodiment curves outward away from the skin. Sheet 10 is bonded to recess in frame 8 so that the back surface is relatively flat. Adhesive 16 is bonded to the rear surface of sheet 10. A releasable substrate protects the adhesive 16 until time of use. FIG. 4 shows an alternate embodiment of the invention wherein the adhesive sheet 10 is removable, i.e. disposable, from frame 8 so that frame 8 can be re-used multiple times while sheet 10 is replaced each time. In this case additional adhesive material is applied to the inside perimeter of sheet 10 as shown by the dotted line 12. An advantage of this embodiment is that the disposable sheet 10 is more easily manufactured than the preferred embodiment that has a frame. An example of the degree to which the Frame 8 curves convexly outward from the thin film sheet 10 is shown in FIG. 5 but this is not meant to limit the scope of this invention by showing just one such degree\angle. FIG. 7 shows an alternate embodiment where the frame portion 34 is only located at the upper edge of sheet 32. The upper edge is where most of the skin distortion occurs in the case of the area under the eyes due to the patch digging into the skin and therefore the need for a frame that surrounds the entire perimeter of sheet 32 is not necessarily imperative. FIG. 7 also shows the alternate embodiment having a slit 36 in frame 34. Slits in the frame may improve a patch's flexibility and make it better fit the area under the eye.

Although the patch shown in the above illustrations is designed to fit below the eyes, it should be understood that the spirit of this patent includes other shaped patches with aforementioned frames that may be used for other areas of the face, e.g. frown lines on the fore-head. While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims. For example, an alternative design might implement the function of the convex shaped frame by having a thick rounded edge instead of the frame itself. Another close alternative would be to use flat patches, some of which are on the market today, with separate frames, disposable or otherwise, placed between the edges of these patches and the skin. But we expect this patent to cover such close alternatives. This patent is intended to cover all anti-wrinkle eye patches with frames, regardless of the frame's shape and size. This patent claim of having a frame around an anti-wrinkle skin patch so that the skin around the edge of the patch is not adversely affected also applies to acne and other such skin patches. Another function of the frame is to make it easy to remove the patch by having something to grip and pull.

What is claimed is:

1. Anti-Wrinkle Patch comprising:
A thin flat flexible sheet die cut to a shape that fits generally in the area directly below the user's eye;
said flat sheet having a non-allergic adhesive substrate on one side; and
said flat sheet perimeter or portion of thereof having a non adhesive convex shaped frame:

2. Anti-Wrinkle Patch as claimed in claim 1 wherein said adhesive substrate contains additional anti-wrinkle ingredients such as anti-oxidants or other agents that claim to reduce wrinkles.

3. Anti-Wrinkle Patch as claimed in claim 1 where said frame portion is fixedly attached to the perimeter of said sheet portion so that the said frame angles away from the skin to prevent any additional wrinkling that might be caused by a regular flat edge.

4. An alternative embodiment of the Anti-Wrinkle Patch as claimed in claim 1 wherein said flexible sheet can be irremovably attached, i.e. disposable, to said frame using standard attachment means so that said frame may be reused in conjunction with a new disposable said flexible sheet.
